(19) United States
(12) Patent Application Publication
(10) Pub. No.: US 2003/0211526 A1
(43) Pub. Date: Nov. 13, 2003

(54) Cross-species nucleic acid probes

(76) Inventors: Jyh-Lyh Juang, Taipei (TW); Chao Agnes Hsiung, Taipei (TW); Chung-Yen Lin, Kaohsiung (TW)

Correspondence Address: FISH & RICHARDSON PC, 225 FRANKLIN ST, BOSTON, MA, 02110, US

(21) Appl. No.: 10/366,823

(22) Filed: Feb. 14, 2003

Related U.S. Application Data
(60) Provisional application No. 60/357,541, filed on Feb. 15, 2002.

(51) Int. Cl. C12Q 1/68; C07H 21/04

(52) U.S. Cl. 435/6; 536/24.3

(57) ABSTRACT

The present invention features a collection of at least four nucleic acid probes. The probes each comprises a segment, the entirety of which hybridizes under low stringency conditions to at least a first gene of a first species and a second gene of a second species, wherein the hybridizing probes correspond to different genes of the first or second species, and the first and the second genes are orthologous to each other. In some embodiments, the entirety of the segment is at least 60% (e.g., 65%, 70%, 80%, 90%, or 95%) identical to the first gene and the second gene.

### RELATED APPLICATION

This application claims priority to U.S. provisional application No. 60/357,541, filed on Feb. 15, 2002, the contents of which are incorporated herein by reference.

### BACKGROUND

Nucleic acid arrays are ordered collections of nucleic acid probes. Specific hybridization of nucleic acids in a sample to the probes can be used to interrogate the composition and abundance of nucleic acids in the sample. There are numerous applications of nucleic acid arrays including analysis of gene expression and of gene polymorphisms.

The nucleic acid sequences of a number of genomes are now available. These genomes include the genomes of humans, model eukaryotic organisms (e.g., Drosophila melanogaster, Caenorhabditis elegans, and Saccharomyces cerevisiae) and bacterial pathogens. A number of genes have been identified that are conserved among metazoans: for example, genes encoding homeobox domains, kinases, and biosynthetic enzymes. Conserved genes and conserved proteins can be identified by aligning amino acid or nucleotide sequences, typically using a computer program. Such an analysis can be extended to the genomic scale. Some exemplary studies are described in Peltonen and McKusick (2001) Science 291: 1224-1229; O'Brien et al. (1999) Science 286: 458-481; and Rubin et al. (2000) Science 287: 2204-2215. Comparative genomics facilitates the elucidation of the evolutionary basis of cellular and developmental processes in different phyla.

### SUMMARY

This invention relates to nucleic acid probes that can be used to analyze samples from a wide spectrum of species. The probes are designed for cross-species hybridization, e.g., by identification of conserved segments among orthologous genes (also termed “orthologs”).

The probes can be attached to an array that can be used for comparative gene analyses. In one aspect, this invention features a collection of at least four (e.g., at least 5, 50, 100, 500, or 1000, or any number between 4 and 10⁴, 10⁵, or 10⁶, or any number beyond 10⁶) nucleic acid probes. The probes each include a segment, the entirety of which hybridizes under low stringency conditions to at least a first gene of a first species and a second gene of a second species. The hybridizing probes correspond to different genes of the first or second species. In each case, the first and the second genes are orthologous to each other. Sequences other than the above-described probes, if any, are not considered as probes, e.g., sequences necessary for experimental controls or for other purposes. In some embodiments, the entirety of the segment is at least 60% (e.g., 65%, 70%, 80%, 90%, or 95%) identical to the first gene and the second gene. In no case is the segment less than 20 nucleotides.

The probes (including DNA and RNA) are nucleic acids that are entirely single stranded or partially single stranded. They can be prepared by a chemical synthesis method, or prepared by a polymerase chain reaction. A probe can be between 20 to 2000, 20 to 800, or, 20 to 500 nucleotides in length. Each probe can be attached to a solid support, e.g., the same solid support or different solid supports. Alternatively, each probe can be free in solution. For example, each probe can be labeled or tagged. Optionally, each probe can be immobilized, e.g., after hybridization. In some embodiments, the probes each include a consensus sequence, or one or more degenerate positions. A “consensus” sequence is a sequence derived from a profile of two or more related sequences. For example, at positions that differ, the most common nucleotide can be included in the consensus. Alternatively, the position can be made degenerate. With respect to a population of probes, a “degenerate” position refers to a position that includes either different nucleotides among the population or an a typical nucleotide (i.e., not adenine, guanine, cytosine, uracil, or thymidine) such as inosine.

The term “segment,” as used herein, refers to a nucleic acid that is conserved among orthologs. The segment is at least 20 nucleotides in length (e.g., 20 to 600, or 20 to 200 nucleotides). A “DNA” refers to the polymeric form of deoxyribonucleotides (adenine, guanine, thymine, or cytosine) in its either single stranded form, or a double-stranded helix. An “RNA” refers to the polymeric form of ribonucleotides (adenine, guanine, uridine, or cytosine) in its either single stranded for, or a double-stranded helix.

In some embodiments, the probe includes a nucleic acid segment of a human disease gene. The gene can encode, for example, an enzyme, a transcription factor, a cell surface protein, or a functional domain. See, e.g., the “Probe Selection” section.

The term “species” refers to a naturally existing population of similar organisms that are given a unique name to distinguish them from all other creatures. A species may include various “serotypes” and “strains,” i.e., the classification of the sub-species, or the descendants of a common species. The first and the second species can be of different genera, different families, different orders, different classes, different phyla, or different kingdoms. The first and the second species can also be different species of the same genus, the same family, the same order, the same class, the same phylum, or the same kingdom. For example, the first and second species might be of different phyla, but the same kingdom, and so forth. The first and the second species can be, independently, a mammal (e.g., a human or a mouse), an invertebrate (e.g., Drosophila or C. elegans), a fungus (e.g., S. cerevisiae), a bacterium (e.g., B. subtilis, E. coli, or P. aeruginosa), or virus (e.g., virus that infects mammalian cells).

In some embodiments, the first species can be a different class from the second species, or a different phylum from the second species. The first and the second species can be, independently, a mammal, an invertebrate, a plant, a fungus, a bacterium, or virus. Clearly, some species are microorganisms, others are multicellular. In one exemplary combination, the first species is a mammal (e.g., human), and the second species is an invertebrate (e.g., Drosophila). Additional examples of species include: Arabidopsis thaliana, Caenorhabditis elegans, Danio rerio, Dictyostelium discoideum, Escherichia coli, Takifugu rubripes, Hepatitis C virus, Mus musculus, Mycoplasma, Oryza sativa, Zea mays, Plasmodium falciparum, Pneumocystis carinii, Rattus, Saccharomyces cerevisiae, Schizosaccharomyces pombe, and Xenopus laevis.

In another aspect, this invention features a method for providing a collection of probes. The method includes for each of a plurality of genes of a first species, identifying a segment that is conserved relative to an orthologous gene from a second species; selecting a sequence from the orthologous gene that is at least 60% (e.g., 65%, 70%, 80%, 90%, or 95%) identical to the conserved segment, and preparing a probe having the selected sequence or a reverse complement of the selected sequence, thereby producing a collection of probes. In some embodiments, the selected sequence is identical to the conserved segment, or identical to a segment of the orthologous gene. In some other embodiments, the selected sequence is a consensus sequence.

This invention also features a collection of probes provided by the just-described method.

In still another aspect, this invention features a method for evaluating a sample. The method includes contacting a sample that comprises nucleic acids from a third species to a collection of nucleic acid probes, wherein (i) each probe comprises a segment, the entirety of which cross-hybridizes under low stringency conditions to at least a first gene of a first species and a second gene of a second species, wherein the collection includes probes corresponding to different genes of the first or second species, and the first and the second genes are orthologous to each other, and (ii) the third species differs from both the first and the second species; evaluating binding of the sample to each of the probes; and for each probe that is bound, inferring the presence, in the sample, of a nucleic acid for a third gene of the third species, the third gene being an ortholog of the first and second gene. In some embodiments, the third species is a different order from either the first or the second species, or a different order from both the first and the second species. The nucleic acids from the third species can be genomic DNA, mRNA, or reversed transcribed mRNA.

In further another aspect, this invention features a substrate that includes a plurality of addresses (e.g., at least 50 addresses, but less than 10000, at least 300 addresses, but less than 5000, or at least 500 addresses, but less than 2000), each address comprising a nucleic acid probe, each probe comprising a segment, the entirety of which cross-hybridizes under low stringency conditions to at least a first gene of a first species and a second gene of a second species, wherein the collection includes probes corresponding to different genes of the first or second species, and the first and the second genes are orthologous to each other. The nucleic acid segment can be between 15 and 600 nucleotides in length, and the first species can be a different order from the second species.

The probes can be covalently, or non-covalently attached to the substrate. The substrate can be a bead, and each expressed sequence can be attached to a different bead. The substrate can be an array, e.g., a glass (e.g., a surface-modified glass), a membrane, or a polymer, and each expressed sequence can be attached to a different address of the same array. The number of the different addresses can be at least 50, but less than 10000, at least 300, but less than 5000, or at least 500, but less than 2000.

Also within the scope of this invention is a collection of nucleic acid probes. Each probe set includes a first and second probe. The first probe includes a first nucleic acid sequence identical to a segment of a first gene of a first species. The second probe includes a second nucleic acid sequence identical to a segment of a second gene of a second species and non-identical to the segment of the first gene. Moreover, the first and second genes are orthologs. Each probe set can further include at least a third probe that has a third nucleic acid sequence identical to a segment of a third gene from a third species. The collection can include a plurality of at least 20, 50, 100, 500 or 1000 probe sets. In one embodiment, the collection includes no more than 20000 or 5000 probe sets.

In another aspect, this invention features a nucleic acid array substrate that includes a plurality of address sets. Each address set includes at least a first and second addresses. The first address has a first nucleic acid probe and the second address has a second nucleic acid probe. The first probe includes a first nucleic acid sequence identical to a segment of a first gene of a first species. The second probe includes a second nucleic acid sequence identical to a segment of a second gene of a second species and non-identical to the segment of the first gene. The first and second genes are orthologs.

The term “address set” merely means a group of addresses (e.g., a group of at least two addresses) that are related by the identity of the probe at each address. The term does not imply a spatial or other physical relationship. In one embodiment, each address of an address set is adjacent to at least another address of the set. Yet, in another embodiment, each address of the set can be located in a different region of the substrate. For example, the substrate can include at least a first and second non-overlapping region. Each first address of a set is located within the first region and each second address of the set is located within the second region.

In further another aspect, the present invention features a method that includes for each of a plurality of expressed sequences of a first species, identifying an orthologous expressed sequence in a second species; selecting a nucleic acid segment of the orthologous expressed sequence; preparing the nucleic acid segment; and attaching the nucleic acid segment to a substrate. The first species can be a different order from the second species. The nucleic acid segment is at least 60% identical between the first and the second species, and is between 15 and 600 nucleotides in length.

In still another aspect, this invention features a method for evaluating a sample. The method includes providing an array, contacting a sample that comprises nucleic acids from a third species to the array, in which the third species differs from both the first and the second species; and evaluating binding of the sample to the array. The array includes a substrate having a first plurality of addresses, each address including a first unique probe that comprises a first nucleic acid segment of a first species. The first nucleic acid segment is at least 60% identical to a gene of the first species and to its ortholog in a second species. The substrate also has a second plurality of addresses, each of which corresponds to each of the first plurality of addresses, and includes a second unique probe that comprises a second nucleic acid segment of the second species. Each of the first and the second nucleic acid segments is between 15 and 600 nucleotides in length, and the first species is a different order from the second species.

The “percent identity” of two amino acid sequences or of two nucleic acids is determined using the algorithm of Karlin and Altschul (1990) Proc. Natl. Acad. Sci. USA 87: 2264-68, modified as in Karlin and Altschul (1993) Proc. Natl. Acad. Sci. USA 90: 5873-77. Such an algorithm is incorporated into the NBLAST and XBLAST programs (version 2.0) of Altschul et al. (1990) J. Mol. Biol. 215: 403-10. BLAST nucleotide searches can be performed with the NBLAST program, score=100, wordlength-12 to obtain nucleotide sequences homologous to the nucleic acid molecules of the invention. BLAST protein searches can be performed with the XBLAST program, score=50, wordlength=3 to obtain amino acid sequences homologous to the protein molecules of the invention. Where gaps exist between two sequences, Gapped BLAST can be utilized as described in Altschul et al. (1997) Nucleic Acids Res. 25(17): 3389-3402. When utilizing BLAST and Gapped BLAST programs, the default parameters of the respective programs (e.g., XBLAST and NBLAST) can be used. See the online site provided by National Center for Biotechnology Information (NCBI) at the National Institute of Health (NIH), Bethesda Md.

As used herein, the term “hybridizes under low stringency conditions,” describes conditions for hybridization and washing. Guidance for performing hybridization reactions can be found in Current Protocols in Molecular Biology, John Wiley & Sons, N.Y. (1989), 6.3.1-6.3.6, which is incorporated by reference. Aqueous and nonaqueous methods are described in that reference and either can be used. Low stringency hybridization conditions referred to herein are as follows: in 6×sodium chloride/sodium citrate (SSC) at about 45° C., followed by two washes in 0.2×SSC, 0.1% SDS at least at 50° C. (the temperature of the washes can be increased to 55° C. for increased stringency conditions). Under the low stringency hybridization conditions, the probes including a consensus sequence, or one or more degenerate positions can hybridize to a first gene of a first species and a second gene of a second species, as described above.

Other exemplary hybridization conditions include: (i) medium stringency hybridization conditions in 6×SSC at about 45° C., followed by one or more washes in 0.2×SSC, 0.1% SDS at 60° C.; (ii) high stringency hybridization conditions in 6×SSC at about 45° C., followed by one or more washes in 0.2×SSC, 0.1% SDS at 65° C.; and (iii) very high stringency hybridization conditions are 0.5M sodium phosphate, 7% SDS at 65° C., followed by one or more washes at 0.2×SSC, 1% SDS at 65° C. Of course, consensus probes and degenerate probes may also hybridize under these, more stringent conditions.

Further, the stringency conditions for hybridization and washing can also be altered by the following factors: For the hybridization conditions: (I) in the presence of formamide in a hybridization buffer, (i) the higher of formamide (ranging from 25 to 50%), the lower of the stringency, (ii) the higher of the hybridization temperature (37° C. to 45° C.), the higher of the stringency, and (iii) the higher of the SSC (3× to 6×), the lower of the stringency; and (II) In the absence of formamide in a hybridization buffer, the higher of the hybridization temperature (ranging from 50° C. to 65° C.), the higher of the stringency. For the washing conditions: (i) the higher of the washing temperature (25° C. to 65° C.) the higher of the stringency, and (ii) the higher of the SSC (0.1× to 2×), the lower of the stringency.

The arrays can be used for a wide variety of applications. One application is a cross species study, especially for those organisms that do not have a comparable amount of sequence information to construct a species-specific microarray. A cross-species array has the further advantage of economy as a cross-species array can be used for more than one species. For example, a cross-species array designed from Drosophila and human sequences can be used to analyze nucleic acid from rodents, fish, snakes, and crustaceans (such as lobster). This approach can also be applied to design cross-species arrays appropriate for plants or microorganisms, e.g., fungi or bacteria. In one implementation, the cross-species array is designed from sequences from extremely divergent species (e.g., species of different kingdoms). The degree of divergence can be tailored according to the desired application.

Some other applications include the analysis of samples to diagnose infectious diseases caused by related pathogens (e.g., the array is a cross-species array designed from microbial sequences), and the analysis of samples to implement plant and animal quarantines. Other of exemplary practical applications of cross-species analyses include: (i) supplying animal models for human genetic diseases; (ii) identifying candidate genes that participate in conserved disease processes; (iii) assessing multi-factorial genetic traits; (iv) identifying adaptations in non-human mammal species that ameliorate maladies homologous to human hereditary and infectious diseases; and (v) developing treatments for veterinary pathologies based on human trials.

In accordance with the present invention there may be employed conventional molecular biology, microbiology, and recombinant DNA techniques within the skill of the art. Such techniques, as well as technique terms, are explained fully in the literature. See, e.g., Ausubel, R. M., ed. (1994) “Current Protocols in Molecular Biology” Volumes I-III; Celis, J. E. ed. (1994) “Cell Biology: A Laboratory Handbook” Volumes I-III; Gait, M. J. ed. (1984) “Oligonucleotide Synthesis”; and Hames, B. D. & Higgins, S. J. eds. (1985) “Nucleic Acid Hybridization.”

This invention also features a collection that includes at least four nucleic acid probes (e.g., at least 5, 50, 100, 500, or 1000, or any number between 4 and 10⁴, 10⁵, or 10⁶, or any number beyond 10⁶), the probes each including a segment, the entirety of which hybridizes under low stringency conditions to at least a first gene of a first sub-species and a second gene of a second sub-species, wherein the hybridizing probes correspond to different genes of the first or second sub-species, and the first and the second genes are orthologous to each other. The first and second sub-species can be two strains or two serotypes. In some embodiments, at least one of the probes comprises at least one degenerate position.

In a further aspect, this invention features a method for evaluating a sample. The method includes contacting a sample that comprises nucleic acids from a third sub-species to a collection of nucleic acid probes, wherein (i) each probe comprises a segment, the entirety of which hybridizes under low stringency conditions to at least a first gene of a first sub-species and a second gene of a sub-second species, wherein the hybridizing probes correspond to different genes of the first or second sub-species, and the first and the second genes are orthologous to each other, and (ii) the third species is the same or different from the first or the second species; evaluating binding of the sample to each of the probes; and for each probe that is bound, inferring the presence, in the sample, of a nucleic acid for a third gene of the third sub-species, the third gene being an ortholog of the first and second gene.

Also within the scope of this invention is a packaged product. The packaged product includes a container, one of the aforementioned collections in the container, and a legend (e.g., a label or an insert) associated with the container and indicating use of the collection for identifying orthologous genes among different species or sub-species.

Other features, objects, and advantages of the invention will be apparent from the description and from the claims.

### DETAILED DESCRIPTION

The present invention relates to a collection of probes that are designed to specifically recognize related nucleic acids from a plurality of species. In one typical implementation the probes are attached to a planar array. The probe collections are designed by first identifying sequences from a first species, identifying sequence orthologs in at least a second species, and then constructing probes.

Probe Selection

To design a collection of probes, sequences of interest from a first species are identified. These sequences can be chosen with respect to the application of interest. For example, to identify pathogens, sequences correlated or associated with pathogenesis can be selected.

In one example, the comparative analysis focuses on human disease genes and their orthologs in other species. A “human disease gene,” herein, refers to a human gene which is naturally polymorphic, and for which one polymorphic allele is correlated with a diagnosable disorder or phenotype. This example of comparative analysis can provide into the mechanism and function of genes associated with diseases. The polymorphic allele can include a mutation, insertion (e.g., trinucleotide repeat expansion), deletion, loss of heterozygosity, or amplification relative to a normal allele, e.g., an allele not correlated or anti-correlated with the diagnosable disorder or phenotype.

Exemplary human diseases for which human disease genes have been identified include cancer, neurological disorders, and endocrine diseases. Human disease genes that are associated with cancer include menin (MEN; multiple endocrine neoplasia type 1), Peutz-Jeghers disease (STK11), ataxia telangiectasia (ATM), multiple exostosis type 2 (EXT2), a second bCL2 family member, a second retinoblastoma family member, and p53-like protein encoded genes. Human disease genes that are associated with neurological disorders include tau (frontotemporal dementia with Parkinsonism), the Best macular dystrophy gene, neuroserpin (familial encephalopathy), genes for limb girdle muscular dystrophy types 2A and 2B, the Friedreich ataxia gene, the gene for Miller-Dieker lissencephaly, parkin juvenile Parkinson's disease), and the Tay-Sachs and Stargardt's disease genes. Orthologs of many of these genes are present in Drosophila (see, e.g., Rubin et al. (2000) Science 287: 2204-2215).

A human disease gene can encode a polypeptide that includes an enzyme, a transcription factor, a cell surface protein, or a functional domain. A “functional domain” includes a polypeptide fragment that can independently participate in an interaction, e.g., an intramolecular or an intermolecular interaction. An intermolecular interaction can be a specific binding interaction or an enzymatic interaction (e.g., the interaction can be transient and a covalent bond is formed or broken).

An analysis can also performed based on genes of different species, and provide insights into understandings of the evolutionary basis of cellular and developmental processes. An probe collections of this invention can include a set of probes relating to genes associated with processes including cell division, cell shape, signaling pathways, cell-cell and cell-substrate adhesion, and apoptosis—determining the developmental outcomes of different embryos. The processes can also include cell-cell interactions, cell polarity and cell movement—determining embryonic gradients, as well as the processes of neuronal signaling and innate immunity. Examples of cell cycle related genes include cyclin A (CycA), CycB, CycB3, CycE, and CycD. Other conserved cyclins that are associated with transcription include CycC, CycH, CycK, and CycT. Examples of orthologs related to cytoskeleton include the tubulin superfamily, such as α-, β-, γ-, δ-, and ε-tubulin, which have been identified in both human and Drosophila. See Rubin et al. (2000) Science 287: 2204-2215.

In another example, the collection of probes is formulated from genes associated with bacterial pathogenesis. The first and second species can be gram-negative and/or gram-positive bacteria for which genes associated with pathogenesis have been identified. Probes to these genes can be used to analyze the pathogenicity of a sample that potentially includes a pathogenesis-associated gene of a third species, different from the first and second species.

Orthologs

Once a group of genes or proteins of interest has been identified from a first or “source” species, an ortholog is identified for each gene or protein from at least a second species. Orthologs are biopolymeric sequences (e.g., nucleic acid or polypeptide sequences) that are found in different species, yet have sequence similarity, and are predicted by a skilled artisan to perform similar functions. An “ortholog” is distinguished from a “homologue” as follows. An ortholog of a first sequence of a first species is the sequence of a second species with the most homology to the first sequence relative to other available sequences of the second species. For example, some sequences are members of large protein families. Even with the same species, a particular sequence may have multiple homologs. However, with respect to a second species, the particular sequence has a distinct ortholog which is the most homologous sequence in the second species. Orthologs can be assigned by comparing numerous sequences to identify the best match-up. See, e.g., Science Oct. 24, 1997; 278(5338): 631-7 and Nucleic Acids Res Jan. 1, 2001; 29(1): 22-28 for some exemplary methods and resource for assigning orthologs based on complete genome coverage.

The second species from which the orthologs are identified can be judiciously chosen in accordance with the desired application. The second species can be as divergent or related to the first species as desired. For example, the second species can be from the same kingdom, phyla, or class. The second species can also be from a different order, class, phyla, or kingdom. The National Center for Biotechnology Information (NCBI; Bethesda Md.) also provides a taxonomy on-line resource that can be used to determine the phylogenetic relationship between two species (Wheeler et al. (2000) Nucleic Acids Res. 28:10-4). The site can be used to identify the full taxonomic classification of a species.

For both nucleic acid and polypeptide sequences, orthologs can be identified by a sequence comparison search. To identify an ortholog of a particular sequence of a first sequence, the first sequence is iteratively compared with each available sequence of the second species. It is, of course, useful, but not necessary to have sequence information for the entire genome of the second species. It can be complementary to the coding strand or to the non-coding strand of a gene.

Information for nucleic acid and protein sequences of a species can be retrieved from publicly available databases. Such databases include, but are not limited to, Online Mendelian Inheritance in Man (OMIM), the Cancer Genome Anatomy Project (CGAP), GenBank, EMBL, PIR, SWISS-PROT, and the like. These databases can be accessed from their on-line facilities using uniform resource locators that are well known to those skilled in the art. Some of these databases contain complete or partial nucleotide sequences for a particular species. In addition, for some species, a large fraction, i.e., close to the entire fraction of the genome is available.

The comparisons can be computed using a computer program such as BLAST. After candidate orthologs are identified, further refinements can be used to identify the ortholog. For example, the sequence match search program, e.g., from the EMBOSS suite of programs (available from UK Medical Reseach Council HGMP Resource Centre, Hinxton, Cambridge, CB10 1SB, United Kingdom), can be used to plot a dot matrix figure of the particular sequence and its ortholog. Based on the match density at a given location, there may be no dots, isolated dots, or a set of dots so close together that they appear as a line. The presence of lines indicates the sequence homology.

Potential orthologs for polypeptides and nucleic acids can be further analyzed to select representative sequences that fit criteria for being conserved sequences. The criteria can be based on cut-off values, referred to as E-values. The lower the E-value represents the better the match. The E-value can be defined depending upon the stringency, or degree of homology desired, as described above. Orthologs can be identified on the basis of nucleic acid or amino acid sequence homology. Typically, nucleic acid sequence homology is used as this homology reflects the utility of nucleic acid probes in hybridization.

Once an ortholog is identified for a particular sequence, both sequences are scanned to identify a conserved segment of about 15 to 300 nucleotides. The length and sequence composition of the segment can be selected such that the sequence has a desired degree of sequence conservation (e.g., E-value or % identity), Tm, specificity, composition, and length. For a particular collection of probes, these parameters can be defined with specific boundaries in order to insure homogeneity in the probe behavior within the collection.

Phylogenetic programs, such as PHYLIP, ClustalW, and Pfam, can be used to compare a family of related sequences and to thereby derive a consensus sequence. The consensus sequence can be used as a probe. In some implementations, at ambiguous positions, a degenerate nucleotide is included.

Probe Construction

Once a conserved segment is identified, a probe corresponding to that segment is constructed. Any of a variety of methods can be used to synthesize the probes. Such methods include chemical synthesis, photolithography, recombinant DNA techniques, and nucleic acid amplification.

In one embodiment, PCR is used to construct the probes. PCR primers are designed that hybridize to the sense and the anti-sense strands of nucleic acids from the first and/or second species. The primers are used to amplify the segment, e.g., by the polymerase chain reaction (PCR). The primers can include a primer pair to amplify a probe from the first species and another primer pair to amplify a probe from the second species. This approach can be extended to obtain orthologous probes from at least a third species. In some cases, the probes sequence are sufficiently identical that a single primer pair suffices. In other cases, probes are only obtained from one of the two species.

In one implementation, asymmetric PCR is used to generate largely single-stranded nucleic acid probes. In another implementation, one of the primers is tagged, e.g., with biotin). After amplification, the extended-tagged primer is isolated from complementary nucleic acid strands to obtain a single-stranded nucleic acid probe.

The amplified nucleic acids are used as probes or formatted for such use. For example, the probes can be immobilized onto a planar substrate to produce a nucleic acid array. The probes can also be attached to a particle (such as a bead). Each probe of the collection can be attached to a different bead. In still another example, the probes are labeled for hybridization experiments. Further, the probes can be packaged in containers (collectively or individually) as a kit.

Arrays

An array of this invention can have many addresses on a substrate. The featured array can be configured in a variety of formats, non-limiting examples of which are described below.

A substrate can be opaque, translucent, or transparent. The addresses can be distributed, on the substrate in one dimension, e.g., a linear array; in two dimensions, e.g., a planar array; or in three dimensions, e.g., a three dimensional array. The solid substrate may be of any convenient shape or form, e.g., square, rectangular, ovoid, or circular. Non-limiting examples of two-dimensional array substrates include glass slides, quartz (e.g., UV-transparent quartz glass), single crystal silicon, wafers (e.g., silica or plastic), mass spectroscopy plates, metal-coated substrates (e.g., gold), membranes (e.g., nylon and nitrocellulose), plastics and polymers (e.g., polystyrene, polypropylene, polyvinylidene difluoride, poly-tetrafluoroethylene, polycarbonate, nylon, acrylic, and the like). Three-dimensional array substrates include porous matrices, e.g., gels or matrices. Potentially useful porous substrates include: agarose gels, acrylamide gels, sintered glass, dextran, meshed polymers (e.g., macroporous crosslinked dextran, SEPHACRYL™, and SEPHAROSE™), and so forth. Still other substrates include surfaces of microfluidic channels and devices, such as “Lab-On-A-Chip™” (Caliper Technologies Corp.).

An array can have a density of at least than 10, 50, 100, 200, 500, 1000, 2000, 10⁴, 10⁵, 10⁶, 10⁷, 10⁸, or 10⁹ or more addresses per cm² and ranges between. In some embodiments, the plurality of addresses includes at least 100, 500, 1000, or 5000 addresses. In some other embodiments, the plurality of addresses includes less than 99, 499, 999, or 9999, addresses. Addresses in addition to the address of the plurality can be disposed on the array. The center to center distance can be 5 mm, 1 mm, 100 μm, 10 μm, 1 μm or less. The longest diameter of each address can be 5 mm, 1 mm, 100 μm, 10 μm, 1 μm or less. Each addresses can contain 1.0 μg, 100 ng, 10 ng, 1 ng, 100 pg, 10 pg, 1 pg, 0.1 pg or less of a capture agent, i.e. the capture probe. For example, each address can contain 100, 10³, 10⁴, 10⁵, 10⁶, 10⁷, 10⁸, or 10⁹ or more molecules of the nucleic acid.

A nucleic array can be fabricated by a variety of methods, e.g., photolithographic methods (see, e.g., U.S. Pat. Nos. 5,143,854; 5,510,270; and. 5,527,681), mechanical methods (e.g., directed-flow methods as described in U.S. Pat. No. 5,384,261), pin based methods (e.g., as described in U.S. Pat. No. 5,288,514), and bead based techniques (e.g., as described in PCT US/93/04145). A capture probe can be a single-stranded nucleic acid, a double-stranded nucleic acid (e.g., which is denatured prior to or during hybridization), or a nucleic acid having a single-stranded region and a double-stranded region. The capture probe can be selected by a variety of criteria, and can be designed by a computer program with optimization parameters. The capture probe can be selected to hybridize to a sequence rich (e.g., non-homopolymeric) region of a nucleic acid. The Tm of the capture probe can be optimized by prudent selection of the complementarity region and length. Ideally, the Tm of all capture probes on the array is similar, e.g., within 20, 10, 5, 3, or 2° C. of one another. A database scan of available sequence information for a species can be used to determine potential cross-hybridization and specificity problems.

Evaluating a Sample

The collections of probes described herein can be used to evaluate a sample, particularly a sample that includes a nucleic acid of a third species that differs from the species used to construct the probes. The evaluation can generate information about the abundance of different nucleic acids in the sample.

For example, if the sample is mRNA or cDNA from a cell or tissue, the information can indicate the level of expression of different genes in the cell or the cells of the tissue. First, RNA is prepared from the sample, e.g., using routine methods. RNA isolation can include DNase treatment to remove genomic DNA and hybridization to an oligo-dT coupled a solid substrate (e.g., as described in Current Protocols in Molecular Biology, John Wiley & Sons, N.Y). The oligo-dT solid substrate is washed and the RNA is eluted. The RNA is then reversed transcribed and, optionally, amplified.

Typically the sample is directly or indirectly labeled. The amplified and/or reverse-transcribed nucleic acid can be labeled, e.g., by the incorporation of a labeled nucleotide. Examples of labels include fluorescent labels, e.g., red-fluorescent dye Cy5 (Amersham) or green-fluorescent dye Cy3 (Amersham), chemiluminescent labels, e.g., as described in U.S. Pat. No. 4,277,437, and colorimetric detection. Alternatively, the amplified nucleic acid can be labeled with biotin and detected after hybridization with labeled streptavidin, e.g., streptavidin-phycoerythrin (Molecular Probes).

The labeled nucleic acid is then hybridized to the cross-species array. In addition, a control nucleic acid or a reference nucleic acid can be contacted to the same array. The control nucleic acid or reference nucleic acid can be labeled with a label other than the sample nucleic acid, e.g., one with a different emission maximum.

Labeled nucleic acids are contacted to an array under judiciously chosen hybridization conditions. Some exemplary specific hybridization conditions include: (i) low stringency hybridization conditions in 6×sodium chloride/sodium citrate (SSC) at about 45° C., followed by two washes in 0.2×SSC, 0.1% SDS at least at 50° C. (the temperature of the washes can be increased to 55° C. for low stringency conditions); (ii) medium stringency hybridization conditions in 6×SSC at about 45° C., followed by one or more washes in 0.2×SSC, 0.1% SDS at 60° C.; (iii) high stringency hybridization conditions in 6×SSC at about 45° C., followed by one or more washes in 0.2×SSC, 0.1% SDS at 65° C.; and (iv) very high stringency hybridization conditions are 0.5M sodium phosphate, 7% SDS at 65° C., followed by one or more washes at 0.2×SSC, 1% SDS at 65° C. Additional guidance for performing hybridization reactions can be found in Current Protocols in Molecular Biology, John Wiley & Sons, N.Y. (1989), 6.3.1-6.3.6. Aqueous and nonaqueous hybridizations methods are described in that reference and either can be used.

After washing, the array is detected to determine the amount of label at each address. Detection can be by image acquisition or other methods. For example, unlabelled hybridized strands can be directly detected using surface plasmon resonance or a change in electrical conductance.

In one implementation, low stringency washing conditions are used initially. A first set of data is acquired to determine the amount of probe bound after the low stringency wash. Then the array is washed using higher stringency conditions, e.g., medium stringency. A second set of data is acquired to determine the amount of probe remaining. This process can be repeated to accrue a series of data sets, each indicating the amount of probe remaining at each address of the array. In cases, where a complete series of data is acquired, the series can be analyzed, e.g., using computer software, to estimate homology between the nucleic acid in a sample and the probe.

A hybridization profile of a sample can be determined based on the extent of hybridization at different addresses of the array. A “hybridization profile” includes a plurality of values, wherein each value corresponds to the level of hybridization of a sample or an amplified sequence to a probe. The value can be a qualitative or quantitative assessment of the level of hybridization.

The profile can be used to characterize the sample. For example, the profile can be compared to a standard profile, e.g., the hybridization profile of a well-studied cell population from a particular species.

In one embodiment, the extent of hybridization at an address is represented by a numerical value and stored, e.g., in a vector, a one-dimensional matrix, or one-dimensional array. The vector x {xₐ, x_{b} . . . } has a value for each address of the array. For example, a numerical value for the extent of hybridization at a first address is stored in the variable xₐ. The numerical value can be adjusted, e.g., for local background levels, sample amount, and other variations. Nucleic acid is also prepared from a reference sample and hybridized to an array (e.g., the same or a different array), e.g., with multiple addresses. The vector y is construct identically to vector x. The sample hybridization profile and the reference profile can be compared, e.g., using a mathematical equation that is a function of the two vectors. The comparison can be evaluated as a scalar value, e.g., a score representing similarity of the two profiles. Either or both vectors can be transformed by a matrix in order to add weighting values to different nucleic acids detected by the array.

In one particular embodiment, the profiles are processed to account for differential hybridization to two probes for orthologous genes from different species. In cases where the sample nucleic acid includes nucleic acid from a third species that differs from the species used to design the probes, an algorithm can be used to determine if nucleic acid in the sample is hybridizing to similar extents to both related probes. A correlation algorithm can be used to quantitatively determine if the two (or more) related probes are detecting a similar signal. The algorithm can also account for phylogenetic proximity of the third species to one of the two species used to design the probes relative to the other. Once a favorable comparison is made, the profile can be reconfigured to account for the inferred abundance of the nucleic acid of the third species based on hybridization to the two orthologous probes. Thus, the use of two probes, one from each ortholog, can be used to improve the quality and reliability of hybridization profiles relative to a single probe.

Profile data can be stored in a database, e.g., a relational database such as a SQL database (e.g., Oracle or Sybase database environments). The database can have multiple tables. For example, raw hybridization data can be stored in one table, wherein each column corresponds to a nucleic acid being assayed, e.g., an address or an array, and each row corresponds to a sample. A separate table can store identifiers and sample information, e.g., the batch number of the array used, date, and other quality control information.

Nucleic acids that are present at similar levels can be identified by clustering data. Nucleic acids can be clustered using hierarchical clustering (see, e.g., Sokal and Michener (1958) Univ. Kans. Sci. Bull. 38: 1409), Bayesian clustering, k-means clustering, and self-organizing maps (see, Tamayo et al. (1999) Proc. Natl. Acad. Sci. USA 96: 2907).

In one particular embodiment, the hybridization profiles represent the expression of genes in a cell. The profiles from such a nucleic acid expression analysis are used to compare samples and/or cells in a variety of states, e.g., as described in Golub et al. ((1999) Science 286: 531). For example, multiple expression profiles from different conditions and including replicates or like samples from similar conditions are compared to identify nucleic acids whose expression level is predictive of the sample and/or condition. Each candidate nucleic acid can be given a weighted “voting” factor dependent on the degree of correlation of the nucleic acid's expression and the sample identity. A correlation can be measured using a Euclidean distance or the Pearson correlation coefficient.

The similarity of a sample expression profile to a predictor expression profile (e.g., a reference expression profile that has associated weighting factors for each nucleic acid) can then be determined, e.g., by comparing the log of the expression level of the sample to the log of the predictor or reference expression value and adjusting the comparison by the weighting factor for all nucleic acids of predictive value in the profile.

An array of human diseases related genes can be used for analysis of differential gene expressions. The human diseases related genes can be identified in both human and Drosophila, as described above. Monitoring the differential gene expression of a nucleic acid in response to the cancer related proteins in a developing organism, e.g., developing Drosophila, can provide insight into understandings of the role of the cancer related proteins in mammals.

The specific example below is to be construed as merely illustrative, and not limitative of the remainder of the disclosure in any way whatsoever. Without further elaboration, it is believed that one skilled in the art can, based on the description herein, utilize the present invention to its fullest extent. All publications recited herein are hereby incorporated by reference in their entirety.

### EXAMPLE

Identification of Orthologs

A nucleic acid array that includes probes to orthologs in both human and Drosophila was constructed. First, a computer program was used to identify nucleic acid probes specific for orthologs that were conserved despite the evolutionary distance between these two species. The BLAST (Basic Local Alignment Sequence Tools, optimized for the x86 LINUX SMP architecture) algorithm was iteratively used to query a non-redundant database (“nr”, which is a database that combines SWISSPROT, TREMBL, and PIR 2001.3) with each predicted translated sequence from Genome Annotation Database of Drosophila (version 2, “gadfly2”). The program was tailored to output sequences of about 150 amino acids in length that aligned to the query sequence with an E-value <e⁻²⁰. The results of this query process indicate that 51% of predicted gene products in Drosophila have at least 30% homology with human proteins. The summary of the results is listed in Table 1 and Table 2.

TABLE 1
Summary of human orthologs for Drosophila genes based on E-value.

| | Number of Human | |
|---|---|---|
| | Orthologs | |
| E value | (Unique human genes) | Total = 14333 (%) |
| 1.00E−180 | 648 (529) | 4.52 |
| 1.00E−150 | 982 (836) | 6.85 |
| 1.00E−120 | 1459 (1295) | 10.18 |
| 1.00E−100 | 1902 (1719) | 13.27 |
| 1.00E−80 | 2545 (2042) | 17.75 |
| 1.00E−60 | 3448 (2704) | 24.05 |
| 1.00E−40 | 4679 (3547) | 32.64 |
| 1.00E−20 | 6555 (4687) | 45.72 |
| 1.00E−10 | 7510 (5258) | 52.38 |

TABLE 2
Summary of human orthologs for Drosophila genes based on
percent identity.

| Protein | Number of Human | |
|---|---|---|
| Identity (%) | Orthologs | % |
| 90% | 69 | 0.48 |
| 80% | 224 | 1.56 |
| 70% | 597 | 4.16 |
| 60% | 1236 | 8.62 |
| 50% | 2372 | 16.54 |
| 40% | 4308 | 30.05 |
| 30% | 7428 | 51.81 |

Constructions of a Human-Fly Evolutionarily Conserved Gene Microarray

For each conserved pair of sequences, one Drosophila and one human, two sets of primers (20 to 25-mers) were designed. One set was used to amplify the Drosophila sequence of the conserved pair. The other set was used to amplify the human sequence of the conserved pair. Each primer set was used in a PCR amplification reaction with appropriate template cDNA or genomic DNA to amplify the Drosophila or human sequence. The amplified segment was generally selected to include an approximately 150 basepair region that was most conserved. After amplification, each amplified segment was spotted onto a coated glass plate to form an array. Each amplified segment from Drosophila was spotted adjacent to the corresponding amplified segment from its human ortholog. A series of different concentrations of positive and negative control probes are also spotted onto the array for normalization purposes. The summary of the results is listed in Table 3.

TABLE 3
The accumulated percentage of conserved coding sequences in
highly identical orthologs* and gadfly2.

| | | Percentage in | Percentage |
|---|---|---|---|
| Identity of | | highly identical | in gadfly2 |
| Conserved Region | Ortholog number | orthologs (%) | (%) |
| 90% | 10 | 0.18 | 0.07 |
| 80% | 333 | 5.96 | 2.32 |
| 70% | 2029 | 36.31 | 14.11 |
| 60% | 5588 | 100 | 38.87 |

*The highly identical orthologs were based on length of orthologs greater than 150 bp.

Probes Containing Degenerate Positions

Oligonucleotide primer sets (see Table 4 below) were used to amplify a 5′UTR region containing 144 basepair in various serotypes of Picornaviridae and a VP1 region containing 478 basepair in Enterovirus 71. The regions were labeled with Cy5-dUTP after PCR amplification. Two degenerated probes (i.e., 5′UTR degenerated probe and VP1 degenerated probe) were prepared, and their sequences were listed in Table 5. The degenerated probes were hybridized to amplified samples from four different serotypes of viruses, i.e., Enterovirus 71, Coxsackievirus A16, Echovirus 30, and Influenza A virus. The results show that the 5′UTR degenerated probe, which was designed to recognize all Enteroviruses, specifically labeled after hybridization with amplified samples from Enterovirus 71, Coxsackievirus A16, Echovirus 30, but not the sample from Influenza A virus. In addition, the VP1 degenerated probe, to differentiate Enterovirus 71 from other Enterovirus serotypes, was tested. As expected, the results showed that the VP1 degenerated probe specifically labeled after hybridization with the amplified sample from Enterovirus 71, but not from Coxsackievirus A16. Thus, a degenerate probe can be designed to specifically detect different or the same serotype of Enterovirus in a microarray analysis.

Enterovirus 71 is the major epidemic pathogen of hand, foot and mouth disease in pan-pacific countries. Due to the multiple serotypes and genogroups of the enterovirus, the sequences between different serotypes or strains of the same species are quite varied. Thus, the design of the oligo probes specific for diagnostic of Enterovirus 71 become complicated. With the concept of orthologue probe, multiple species' sequences of the Picornavirus were aligned to design pan-Picornavirus probes. In addition, probes specific to the Enterovirus 71 that corresponding to the aligned multiple strains' sequences of Enterovirus 71 were designed. As described above, the probes carried multiple degenerated sites in their sequences to represent the collection of the target viruses. The binding efficacy and specificity of these orthologous probes to the Enterovirus 71 targets were also shown above.

TABLE 4

Specific oligonucleotide primer sets for
PCR amplification of Picornaviridae.

| | | | | | |
|---|---|---|---|---|---|
| Region¹ | Specific Serotypes² | Primer | Sequence | Length (bp) | Amplicon (bp) |

| | | | | | |
|---|---|---|---|---|---|
| 5′-UTR | Most of | 5-UTR-s | CCCCTGAATGCGG | 13 | 144 |
| | Picornaviruses | | (SEQ ID NO. 1) | | |
| | | 5-UTR-a | GTCACCATAAGCAG | 17 | |
| | | | CCA | | |
| | | | (SEQ ID NO. 2) | | |
| VP1 | Most of EV71 | VP1-s | GAGAGTATGATTGA | 14 | 478 |
| | | | (SEQ ID NO. 3) | | |
| | | VP1-a | GGTCTTTCTCCTGT | 22 | |
| | | | TTGTGTTC | | |
| | | | (SEQ ID NO. 4) | | |

¹Abbreviation:
5′-UTR is 5′-terminal untranslated region;
VP is viral genome protein.
²Abbreviation:
EV71, Enterovirus 71;
CVA16, Coxsackievirus A16.

TABLE 5

Degenerated probes used for detect virus targets in the sample.

| | | | | |
|---|---|---|---|---|
| | | | Degenerated | |
| Probe¹ | Specific Serotypes | mer | Site | Sequences² |

| | | | | |
|---|---|---|---|---|
| 5′-UTR | Most of | 62 | 7 | TGTCGTAAYGSGCAASTCYGYRGCGGAACCG |
| | Picornaviruses | | | ACTACTTTGGGTGTCCGTGTTTCMTTTTATT |
| | | | | (SEQ ID NO. 5) |
| VP1-s | Most of EV71 | 73 | 8 | TCACCYGCGAGCGCYTAYCARTGGTTTTAYG |
| | | | | ACGGGTAYCCCACRTTYGGTGAACACAAACA |
| | | | | GGAGAAAGACC |
| | | | | (SEQ ID NO. 6) |

¹Abbreviation:
5′-UTR is 5′-terminal untranslated region;
VP is viral genome protein;
PC is positive control;
NC is negative control.
²Symbols for nucleotide:
M (amino) is A and C;
R (purine) is G or A;
S (strong) is G or C;
W (weak) is A or T;
Y (pyrimidine) is T or C.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

What is claimed is:

1. A collection comprising at least four nucleic acid probes, the probes each including a segment, the entirety of which hybridizes under low stringency conditions to at least a first gene of a first species and a second gene of a second species, wherein the hybridizing probes correspond to different genes of the first or second species, and the first and the second genes are orthologous to each other.

2. The collection of claim 1, wherein the entirety of the segment is at least 60% identical to the first gene and the second gene.

3. The collection of claim 2, wherein the entirety of the segment is at least 70% identical to the first gene and the second gene.

4. The collection of claim 1, wherein the probes are attached to the same substrate.

5. The collection of claim 1, wherein the first and the second species are of different genera.

6. The collection of claim 1, wherein the first and the second species are of different families.

7. The collection of claim 1, wherein the first and the second species are of different classes.

8. The collection of claim 1, wherein the first and the second species are of different phyla.

9. The collection of claim 1, wherein the first and the second species are of different kingdoms.

10. The collection of claim 1, wherein the first and the second species are different species of the same genus.

11. The collection of claim 1, wherein the first and the second species are different species of the same family.

12. The collection of claim 1, wherein the first and the second species are different species of the same class.

13. The collection of claim 1, wherein the first and the second species are different species of the same phylum.

14. The collection of claim 1, wherein the first and the second species are different species of the same kingdom.

15. The collection of claim 1, wherein the probes are between 20 and 500 nucleotides in length.

16. The collection of claim 1, wherein each of the probes comprise a consensus sequence.

17. The collection of claim 1, wherein each of the probes comprises a degenerate position.

18. A method for producing a collection of probes, the method comprising:
for each of a plurality of genes of a first species, identifying a segment that is conserved relative to an orthologous gene from a second species;
selecting a sequence from the orthologous gene that is at least 60% identical to the conserved segment, and
preparing a probe having the selected sequence or a reverse complement of the selected sequence, thereby producing a collection of probes.

19. The method of claim 18, wherein the selected sequence is identical to the conserved segment.

20. The method of claim 18, wherein the selected sequence is identical to a segment of the orthologous gene.

21. The method of claim 18, wherein the selected sequence is a consensus sequence.

22. A collection of probes provided by the method of claim 18.

23. A method for evaluating a sample, the method comprising:
contacting a sample that comprises nucleic acids from a third species to a collection of nucleic acid probes, wherein (i) each probe comprises a segment, the entirety of which hybridizes under low stringency conditions to at least a first gene of a first species and a second gene of a second species, wherein the hybridizing probes correspond to different genes of the first or second species, and the first and the second genes are orthologous to each other, and (ii) the third species differs from both the first and the second species;
evaluating binding of the sample to each of the probes; and
for each probe that is bound, inferring the presence, in the sample, of a nucleic acid for a third gene of the third species, the third gene being an ortholog of the first and second gene.

24. The method of claim 23, wherein the first and second species are of different genera.

25. The method of claim 23, wherein the first and second species are of different families.

26. The method of claim 23, wherein the first and second species are of different classes.

27. The method of claim 23, wherein the first and second species are of different phyla.

28. The method of claim 23, wherein the first and second species are of different kingdoms.

29. The method of claim 23, wherein the first and the second species are different species of the same genus.

30. The method of claim 23, wherein the first and the second species are different species of the same family.

31. The method of claim 23, wherein the first and the second species are different species of the same class.

32. The method of claim 23, wherein the first and the second species are different species of the same phylum.

33. The method of claim 23, wherein the first and the second species are different species of the same kingdom.

34. A substrate comprising a plurality of addresses, each address comprising a nucleic acid probe, each probe comprising a segment, the entirety of which hybridizes under low stringency conditions to at least a first gene of a first species and a second gene of a second species, wherein the hybridizing probes correspond to different genes of the first or second species, and the first and the second genes are orthologous to each other.

35. The substrate of claim 34, wherein the substrate is comprises glass, a membrane, a polymer, or a bead.

36. The substrate of claim 35, wherein the substrate is surface-modified glass.

37. A collection comprising at least four nucleic acid probes, the probes each including a segment, the entirety of which hybridizes under low stringency conditions to at least a first gene of a first sub-species and a second gene of a second sub-species, wherein the hybridizing probes correspond to different genes of the first or second sub-species, and the first and the second genes are orthologous to each other.

38. The collection of claim 37, wherein the first and second sub-species are two serotypes.

39. The collection of claim 37, wherein the first and second sub-species are two strains.

40. The collection of claim 37, wherein at least one of the probes comprises at least one degenerate position.
